(19) United States
(12) Patent Application Publication
(10) Pub. No.: US 2008/0057531 A1
(43) Pub. Date: Mar. 6, 2008

(54) SYSTEMS, KITS, AND METHODS FOR DETECTING CARIOGENIC BACTERIA AND ASSESSING RISK OF DENTAL CARIES

(75) Inventors: Curtis A. Machida, Portland, OR (US); Tom Maier, Portland, OR (US)

Correspondence Address: KLARQUIST SPARKMAN, LLP, 121 SW SALMON STREET, SUITE 1600, PORTLAND, OR, 97204, US

(73) Assignee: Oregon Health and Science University

(21) Appl. No.: 11/835,411

(22) Filed: Aug. 7, 2007

Related U.S. Application Data
(60) Provisional application No. 60/821,672, filed on Aug. 7, 2006.

(51) Int. Cl. C12Q 1/02

(52) U.S. Cl. 435/32; 435/29

(57) ABSTRACT

Provided are systems, kits, and methods for the detection and identification of cariogenic bacteria in dental plaque and for assessing the risk in a patient of development dental caries based upon the presence of cariogenic bacteria.

### CROSS REFERENCE TO RELATED APPLICATION

This application claims the benefit of the earlier filing date of U.S. Provisional Application No. 60/821,672, filed Aug. 7, 2006, which is incorporated herein by reference in its entirety.

### FIELD

The present disclosure relates to dentistry. More specifically, disclosed herein are systems, kits, and methods for detecting the presence of cariogenic bacteria in oral samples, such as dental plaque and/or saliva and for assessing the risk in a patient of developing dental caries based upon the presence of one or more species of cariogenic bacteria.

### BACKGROUND

Streptococci, including Streptococcus mutans and Streptococcus sobrinus, and Lactobacilli are the major microbiological determinants for dental caries. Currently available tests for evaluating the risk of developing dental caries are based upon the growth of Mutans Streptococci or Lactobacilli species on selective agars. For example, the use of Mitis salivarius medium with sucrose and bacitracin has been reported for the isolation of Mutans Streptococcus (Gold et al., “A Selective Medium for streptococcus Mutans” Arch. Oral Biol. 18:1357-1364 (1973)) and Rogosa medium with acetate and low pH has been reported for the isolation of Lactobacillus (Rogosa et al., “A Selective Medium for Isolation of Oral and Faecal Lactobacilli” J. Bact. 62:132-133 (1951)). These tests find limited utility, however, because they are only semi-quantitative and require extended periods of time (typically between 48 and 72 hours) for development of visible bacterial colonies.

There remains a need in the art for systems, kits, and methods for achieving the rapid and quantitative selection and detection of the major microbiological determinants for dental caries.

### SUMMARY

The present disclosure addresses these and other related needs by providing, inter alia, systems, kits, and methods for the rapid and quantitative selection and detection of one or more cariogenic microorganism.

Within certain embodiments, provided are methods for the selection and identification of one or more cariogenic microorganism from an oral sample wherein the cariogenic microorganism includes a Streptococcus and/or Lactobacillus species, particularly wherein the Streptococcus and/or Lactobacillus species is associated with an increased risk in a patient of developing dental caries. Such methods comprise selecting for growth and/or survival of one or more Streptococcus and/or Lactobacillus species that is associated with the development of dental caries and detecting the presence of the one or more Streptococcus and/or Lactobacillus species.

In a further embodiment, the disclosure provides for assessing the risk of a subject developing dental caries associated with the presence of one or more cariogenic microorganism. In one aspect, wherein the cariogenic microorganism is known to be associated with the occurrence of dental caries, an extent of growth (for example a quantity) of such a microorganism can be correlated with a risk of dental caries.

Methods disclosed herein typically may be completed within from about 20 minutes to about 12 hours of incubation time. Longer incubation times, such as from about 12 hours to about 24 hours or even longer of course can be used, but typically are not required. In certain embodiments the incubation time is typically from about 40 minutes to about 8 hours. Thus, such methods may be usefully employed for rapidly quantifying the major microbiological determinants of risk for the development of dental caries and will, consequently, find utility in the provision of oral health.

Within certain aspects of the presently disclosed methods, selective growth and/or survival of one or more Streptococcus and/or Lactobacillus species may be achieved by employing one or more selective growth medium or other suitable growth condition such that the growth and/or viability of one or more bacterial species other than the one or more caries associated Streptococcus and/or Lactobacillus species is inhibited. For example, the growth condition provides a selective advantage to the caries associated species as compared to non-caries-associated species. In certain embodiments, the growth medium comprises one or more selection agent, such as a selective growth medium that selectively promotes the growth of the caries-associated species, or an antibiotic agent against which one or more cariogenic Streptococcus and/or Lactobacillus species is resistant and to which the one or more non-cariogenic bacterial species is sensitive. Exemplified herein are growth media that employ one or more of bacitracin (MSSB), and/or Rogosa Medium selection. In one embodiment selection is enhanced by the addition of an accelerant that increases the growth rate of the cariogenic bacteria, such as an accelerant that increases the growth rate of cariogenic bacteria that have been selected by the growth medium.

In a particular embodiment the detection of one or more cariogenic Streptococcus and/or Lactobacillus species is achieved, for example, by employing an ATP bioluminescence assay, such as an ATP bioluminescence swab test. In one aspect, the cariogenic bacteria are quantified, for example by bioluminescence detected by the assay. The quantification of the cariogenic bacteria provides a relative indication of the quantity of cariogenic bacteria in the mouth of the subject, which in turn serves as an indicator of cariogenic risk in the subject. The quantification can be determined, for example, by reference to a standard comparison value for a population of subjects who are known to either have or not have an increased risk of dental caries. Alternatively, the reference can be a value obtained from a person who is known not to be at increased risk of dental caries.

Because the disclosed methods may be performed rapidly and quantitatively, they are useful for providing real-time results for risk of dental caries directly to the subject at the time of examination. Thus, one disclosed embodiment provides for the evaluation of a therapeutic regimen, for example, by assaying for cariogenic bacteria before and after treatment. Alternatively, a subject can be selected for caries prevention treatment or other appropriate therapy if the assay indicates an increased risk of dental caries in the subject.

In certain embodiments, kits are provided that may be advantageously employed for the detection of Streptococci or Lactobacilli species that are associated with the development of dental caries.

The foregoing and other objects, features, and advantages of the invention will become more apparent from the following detailed description, which proceeds with reference to the accompanying figures.

### BRIEF DESCRIPTION OF THE DRAWINGS

FIG. 1 illustrates the correlation between ATP concentration (using ATP chemical standards) and relative light units obtained from a CariScreen ATP meter and a Veritas luminometer.

FIG. 2 illustrates the growth curve relationships of several oral streptococci species and ATP content.

FIG. 3 illustrates the relationship between bacitracin concentration and effect on growth of several oral streptococci species as measured by ATP luminescence.

FIG. 4 illustrates the effect of sucrose as a potential accelerant of growth of four different oral streptococci, increase of four different streptococci strains, including measurement of adherent bacteria, in response to varying sucrose concentrations.

FIG. 5 illustrates the bacitracin-based selection for the highly cariogenic S. mutans species in the presence of non-mutans streptococci.

FIG. 6 demonstrates that oral samples can be incubated in bacitracin-containing media to select for the highly cariogenic S. mutans species, and ATP-driven bioluminescence can be used to measure these S. mutans bacteria.

### DETAILED DESCRIPTION

The etiology of dental caries is associated with the acid by-products of bacterial metabolism. Dental caries is a disease characterized by dissolution of the mineral portion of the tooth. As caries progresses, destruction of tooth enamel and dentine occurs followed by inflammation of pulp and periapical tissues. The production of these byproducts is related to a group of aciduric oral microorganisms collectively referred to as cariogenic bacteria. The mutans streptococci, a cluster of acidogenic, dental plaque-inhabiting streptococcal species and various Lactobacillus species are considered the principal causative agents of caries. Important microorganisms in this group that are found in humans include the mutans streptococci species, S. mutans, S. rattus, S. cricetus, S. sobrinus, S. ferns, S. macacae, S. downei and Lactobacillus casei. Of these species it currently is understood that S. mutans and S. sobrinus are of the greatest significance in terms of human caries.

### I. INTRODUCTION

Traditionally, the presence of a particular bacterium is assayed by growing it under conditions that select for its growth, for example, growth on selective media. The majority of currently available tests for evaluating the risk of developing dental caries are based upon the ability of mutans Streptococci or Lactobacilli species to uniquely grow on certain selective agars or media. For example, the use of Mitis salivarius medium with sucrose and bacitracin for the isolation of Mutans Streptococus (Gold et al., “A Selective Medium for Streptococcus Mutans” Arch. Oral Biol. 18:1357-1364 (1973)) and Rogosa medium with acetate and low pH for the isolation of Lactobacillus (Rogosa et al., “A Selective Medium for the Isolation of Oral and Faecal Lactobacilli” J. Bact. 62:132-133 (1951)) form the basis of the most popular currently available assays for highly cariogenic oral bacteria. Although popular, and commercially successful, these tests have limited utility, because require extended periods of time (typically between 48 and 72 hours) for development of visible bacterial colonies.

Another way to measure bacterial numbers is to quantitate the ATP that they produce. Bacteria, like all living cells, produce ATP to drive their enzymatic processes. Measuring this ATP is a fast, although indirect, way to quantitate bacterial presence. ATP is typically measured by utilizing enzymes that require ATP to modify their substrates. These ATP-driven enzymatic reactions are generally designed to result in a color change or bioluminescence, which can then be quantitated. The use of ATP bioluminescence as a quantitative measure of microorganisms was first developed in the 1960s for use in spacecraft clean-rooms. Within the last several years ATP swab tests have been described for hygiene monitoring in the food industry (Kikkoman Corp., Noda-shi, Japan). More recently, Oral BioTech (Albany, Oreg.) developed an ATP bioluminescence swab test for detecting and quantifying “decay-causing bacterial biofilm” in dental plaque samples. The Oral BioTech system (referred t ‘CariScreen’) nonspecifically measures ATP produced by all the oral microorganisms sampled in the dental plaque biofilm without discrimination of those bacteria that are associated with the development of dental caries in a patient. The oral microflora is very diverse, usually with a minority of the total microflora population composed of cariogenic Streptococci or Lactobacilli strains. Thus, without prior selection, the CariScreen system is incapable of specifically quantitating the amount of the highly cariogenic bacteria in a heterogenous microbial population within dental plaque.

Disclosed herein is a method for measuring the determinants of increased risk of dental caries by the selective growth of cariogenic Streptococcus and/or Lactobacillus species. Accordingly, disclosed herein are systems, kits, and methods that are based upon a two-step process whereby in a first step selective growth and/or survival of one or more cariogenic bacterial species from an oral sample is achieved, and in a second step those cariogenic bacterial species are specifically detected and/or quantified thereby permitting a rapid assessment of the associated risk of developing dental caries.

The disclosure of all publications, patents, and patent applications cited herein, whether supra or infra, are hereby incorporated by reference in their entirety. However, in the event of any conflict between the incorporated disclosures and the present specification, the present specification will control. The present invention will be better understood through the detailed description of the specific embodiments, each of which is described in detail herein below. As used in this specification and the appended claims, the singular forms “a,” “an” and “the” include plural references unless the content clearly dictates otherwise.

As used herein, the term “oral” refers to any portion of the oral cavity, including the tongue, gum, teeth including both supergingival and subgingival surfaces and other surfaces in the mouth. An “oral sample” is a sample taken from the oral cavity that potentially contains cariogenic bacteria that can be selected (for example by culturing in a selective culture medium that promotes the growth of cariogenic bacteria and/or inhibits the growth of non-cariogenic bacteria).

“Plaque” refers to the biofilm that forms in vivo on tooth surfaces in the mouth.

“ATP” refers to adenosine triphosphate.

The term “cariogenic bacteria” refers to bacteria associated with an increased risk of developing dental caries in a subject whose oral fluids contain such bacteria, for example above a cariogenic threshold. The cariogenic threshold can be set by those of skill in the art practicing the methods in view of the present disclosure. For example a user can determine the cariogenic threshold based on a risk of cariogenesis. By way of example, cariogenic bacteria include mutans streptococci and Lactobacillus, including, without limitation, S. mutans, S. rattus, S. cricetus, S. sobrinus, S. ferns, S. macacae, S. downei and Lactobacillus casei. In particular, S. mutans and S. sobrinus currently are believed to be of the greatest significance in terms of human caries.

“Quantitating” cariogenic bacteria includes methods of semi-quantitative determinations or assessments of relative quantity compared to controls. “Quantitating” does not require or imply a determination of an absolute quantity (although it does not exclude it).

### II. SELECTION OF CARIOGENIC STREPTOCOCCUS OR LACTOBACILLI SPECIES

In one embodiment, the disclosed methods for detecting cariogenic bacteria include a first selection step, for example by culturing in a selective culture medium, which provides for the selective growth of cariogenic species relative to other oral microflora present in an oral sample. Selection includes contacting the oral sample with at least one selection agent, for example a selection agent in the culture medium that favors the growth of a target organism or which inhibits growth of a non-target organism. In principle any selection agent that favors the growth of cariogenic Streptococcus or Lactobacilli over less cariogenic species can be used as described herein to select for such cariogenic bacteria. For example, a selection agent may retard the growth of non-cariogenic and minimally cariogenic oral microflora more than it retards the growth of cariogenic Streptococcus and/or Lactobacilli. Exemplary selective agents include pore forming antibiotics, such as peptide-based pore forming antibiotics. In particular embodiments, bacitracin and/or a mutacin exhibit high specificity in selecting for cariogenic Streptococcus and/or Lactobacilli. For example, such agents may be employed for the pre-selection of Streptococcus mutans from other bacteria from an oral sample obtained from a subject. Streptococcus mutans are resistant to both bacitracin and mutacins whereas most other oral microflora, including dental plaque bacteria such as non-mutans Streptococci, are susceptible to and substantially unable to grow in the presence of these selective agents. In certain embodiments plural selection agents may be used either in series or at the same time to select for cariogenic bacteria. In one embodiment useful for selecting for cariogenic Streptococci, the selection agent includes an inhibitory amount of bacitracin, and a selection medium optionally includes mitis salivarius plus sucrose sufficient to enhance or accelerate the growth of cariogenic Streptococci (for example from about 0.1% to about 1% or greater concentrations of sucrose) and bacitracin medium (MSSB). The use of bacitracin (in MSSB) for inhibiting the growth of oral microorganisms, with the exception of Streptococci mutans, has been described. Gold et al., A Selective Medium for Streptococcus mutans, Arch. Oral Biol. 18:1357-1364 (1973).

The selection agent optionally comprises other components that enhance the selection step of the disclosed methods. For example, in one embodiment a selection agent includes an accelerant. Such accelerants enhance the growth of cariogenic bacteria.

In one embodiment the selection agent comprises a mutacin. Any mutacin that selectively favors the growth of cariogenic bacteria may be employed in the presently disclosed systems, kits and methods for detecting cariogenic bacteria. In particular, the mutacins from Streptococcus mutans UA159 are useful in the disclosed embodiments employing a sufficient amount of a mutacin as a selection agent or as a component of a selection agent.

Mutacins are post-translationally modified peptides known as lantibiotics (lanthionine-containing antibiotics; see Smith et al., Biochemistry 42:10372-10384 (2003)), which are produced by many lactic acid producing, gram-positive bacteria. Mutacins have broad anti-bacterial activity against gram-positive bacteria. Mutacins isolated from strain UA159 are inhibitory for Streptococci with the exception of Streptococcus Mutans and Streptococcus sobrinus (Hillman et al., “Genetic and Biochemical Analysis of Mutacin 1140, a Lantiobiotic from Streptococcus Mutans” Infection and Immunity 66:2743-2749 (1998) and Hale et al., “Bacteriocin (Mutacin) Production by Streptococcus Mutans Genome Sequence Reference Strain UA159: Elucidation of the Antimicrobial Repertoire by Genetic Dissection” Applied and Environmental Microbiology 71:7613-7617 (2005).

Mutacins are rapidly bactericidal by forming membrane pores and disrupting the cytoplasmic membrane creating an efflux of ions, ATP, and other cellular components. The genetic sequence of Streptococcus mutans UA159 recently was published as part of the microbial genome project, and has been established as a reference strain.

Because of their rapid kill kinetics, mutacins are capable of creating a 2 to 3-log reduction in cell number within 20-40 minutes. Thus, in one embodiment, mutacins, such as those from UA159 can be used as a pre-selection tool with a detection assay, such as an ATP bioluminescence test to qualitatively and quantitatively measure oral mutans Streptococci from oral samples.

Because mutacins cause the release of ATP from susceptible bacteria, one aspect of a method employing a mutacin in the selective medium includes a step of eliminating extracellular ATP released from mutacin-inhibited bacteria, including mutacin-inhibited streptococci, by incubating the extracellular ATP with, for example, adenosine phosphate deaminase and apyrase. Indeed, removal of extracellular ATP from samples prior to cell lysis may be beneficial in other embodiments. One of skill in the art can remove such extracellular ATP according to the methods described in U.S. Pat. No. 6,200,767 to Sakakibara et al.

As demonstrated herein, Lactobacilli may be selected by utilizing a selective medium, such as Rogosa Medium. Lactobacilli grow well in Rogosa Medium whereas most of the other oral bacteria, including dental plaque bacteria, such as streptococcal flora found in dental plaque, are unable to grow in this medium that provides a pH and nutrients that selectively support the growth of Lactobaccilli. Rogosa Medium is disclosed, for example, in Caufield et al. Caries Research 2007; 41:2-8 and in Rogosa et al., “A Selective Medium for Isolation of Oral and Faecal Lactobacilli” J. Bact. 62:132-133 (1951) Rogosa Medium selection for Lactobacillus species can occur within hours.

In one embodiment the disclosed selection agents are effective to yield a bacterial population of about 90% of mutans versus non-mutans streptococci in an oral sample. In particular, the selection agents are effective to yield a 90% non-mutans streptococci sample within from about 20 minutes to about four hours, such as from about 12 hours to about 24 hours, or from about 40 minutes to about three hours, and in particular from about one to about two hours. Typically, a 90% population of mutans streptococci is sufficient to quantify the cariogenic bacteria present in an oral sample. However, in certain embodiments a population greater than about 75%, such as greater than about 80% mutans streptococci is used. Such lower selection rates can be obtained in brief time periods, such as less than about two hours, less than about 90 minutes, or even less than about 45 minutes.

Table 1 discloses particular examples of oral streptococci and lactobacilli, with American Type Culture Collection identifiers, evaluated herein. Although specific ATCC deposit numbers are provided in Tablel, the deposited organisms have been selected as representative examples of the bacterial species, and the species listed are not to be limited to the deposited organism unless context clearly indicates otherwise.

TABLE 1

| Indicator Bacterial (Number of Strains | ATCC |
|---|---|
| Tested) | No. |
| Streptococcus mutans | 700610 |
| | (UA159) |
| Streptococcus mutans | 25175 |
| Streptococcus sobrinus | 33478 |
| Streptococcus sanguis | 10556 |
| Streptococcus oralis | 35037 |
| Streptococcus gordonii | 10558 |
| Streptococcus salivarius | 25975 |
| Lactobacilli acidophilus | 4356 |
| Lactobacilli casei | 334 |
| Lactobacilli fermentum | 14931 |

### III. DETECTION/DETERMINATION OF CARIOGENIC BACTERIA

Following the specific selection of one or more cariogenic bacterial species, systems, kit, and methods disclosed herein are designed to detect and determine (for example quantify) the one or more cariogenic bacterial species. “Determining” a bacterial species refers to detecting a presence and an indication of an amount of the bacteria, such as a quantitative or semi-quantitative assay.

In one embodiment disclosed herein the detection, determination and/or quantitation of cariogenic bacteria, such as Streptococcus mutans is used to guide treatment of a subject. For example, because of the rapid results obtained in certain disclosed embodiments, cariogenic bacteria can be determined in a clinical setting by, for example a dentist or other health professional. In one aspect the disclosed methods can be used for determination outside of the laboratory to provide for a classification of patients into categories for treatment, such as patients of high, intermediate and low dental caries risk. The determination that a particular patient is at risk, would allow preventative measures to be taken to reduce the patient's susceptibility to dental caries, such as the use of professional teeth cleaning, variation in diet, fluoride treatment, treatment of lesions, direct antibacterial therapy such as the use of chlorhexidine or antibiotics and other preventative or therapeutic treatment known to health care professionals in the field.

One exemplary method for detecting bacteria following the step of selection employs an ATP bioluminescence methodology, which is described in greater detail herein. For example, systems, kits, and methods disclosed herein may utilize a swab, tube, and bioluminescence detector. One exemplary method for detecting bioluminescence is described by U.S. Pat. No. 6,200,767 to Sakakibara et al., which is incorporated herein by reference in its entirety.

Thus, within certain embodiments, the present systems, kits, and methods provide that microorganisms are collected from a subject using a sterile swab, which is inserted into a first solution comprising a selective medium. After a selection time sufficient to substantially reduce the population of non-cariogenic bacteria relative to cariogenic bacteria, the bacteria are quantitated. The bacteria can be quantitated by any conventional means, such as by microscopic instrumentation with a hematocytometer, turbidimetry, gravimetry, packed volume, and/or colony counting. However, specifically contemplated herein is a method for quantitating the bacteria, wherein following a selection time, the first solution is contacted with a second solution comprising a cell-lysis solution containing luciferin, luciferase and Mg⁺² is subsequently contacted with the first solution whereby microorganisms from the swab and luciferin-containing solution are mixed. The quantity of visible light released from the luciferin reaction, driven by the ATP originating from the collected microorganisms, may then be measured, for example by using a hand-held luminometer. In one embodiment, the first solution is treated with an ATP eliminating reagent to reduce an amount of extracellular ATP contained in the first solution prior to subjecting the first solution to cell lysis. Using this method, the extracellular ATP in a sample can be eliminated or reduced to a minimal level, which reduces the background luminescence produced by measurement of extracellular ATP not related to the presence of cariogenic bacteria. Examples of ATP eliminating reagents include adenosine phosphate deaminase alone or in combination with an enzyme such as apyrase, alkaline phosphatase, acid phosphatase, hexokinase and adenosine triphosphatase.

Reagent kits for measuring ATP via luminescence amount using a luciferin-luciferase containing luminescent reagent are commercially available, as luminometers for measuring luminescence. The presently disclosed methods can be carried out with commercially available kits and apparatus to measure ATP contained in a subject microorganism via luminescence.

By way of example, the luciferin-luciferase containing luminescent reagent (reagent for measuring ATP) can include 10 mM magnesium sulfate (Mg ion), 0.30 mM D-luciferin (luminescent material), 1.0 mM EDTA (stabilizer), 1.0 mM dithiothreitol (stabilizer), 0.51 mg/ml luciferase (from Genji firefly), 0.2% bovine serum albumin (BSA) (stabilizer), in 50 mM HEPES buffer (pH 7.8). Of course this luminescent reagent composition is merely exemplary.

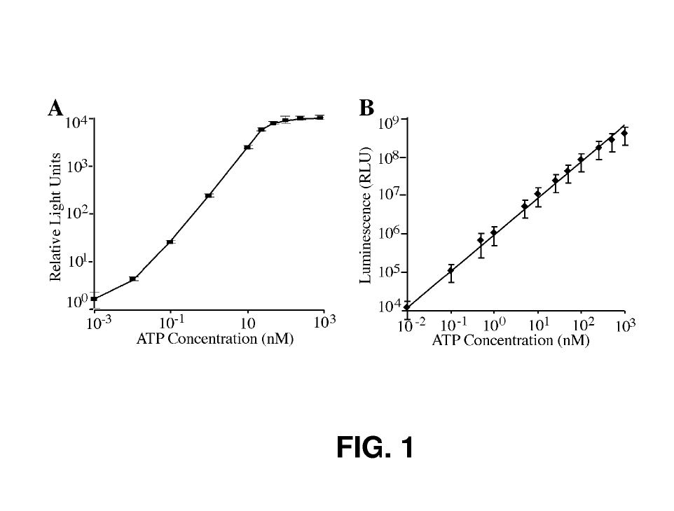

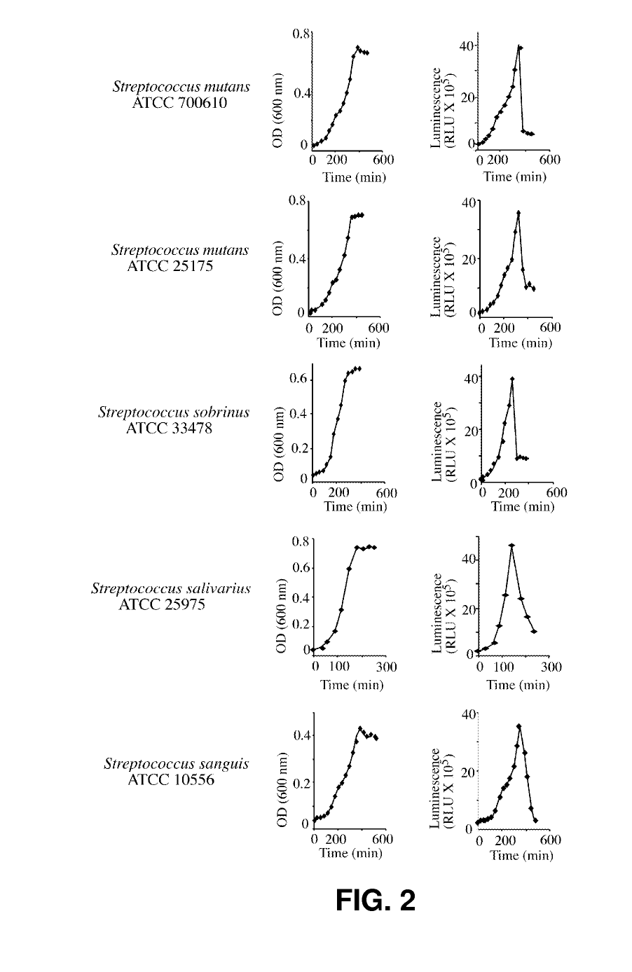

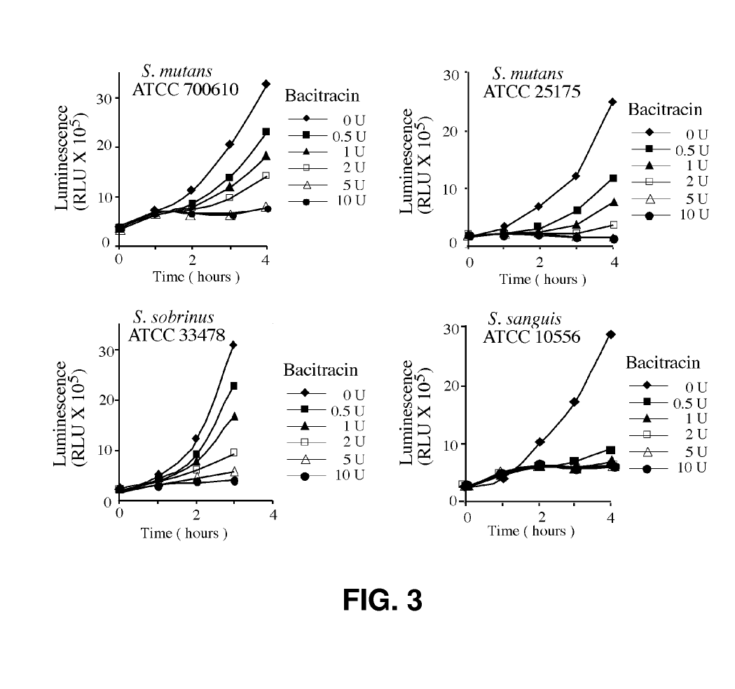

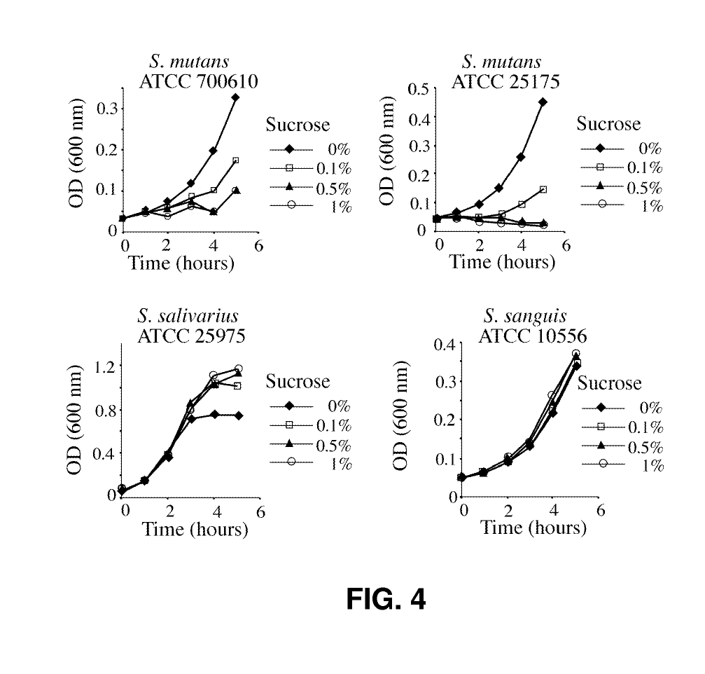

With reference to FIG. 1, the linear portion of the curves in each graph illustrates a range of measurements that can be used to accurately correlate ATP concentration and relative light units. FIG. 2 illustrates that the relationship of ATP content and viable cell number is linear during exponential phase of growth. However, ATP content becomes diminished during transition from exponential phase to lag phase of growth. The data illustrated in FIG. 3 demonstrate the rate of bacitracin selection on cariogenic and non-cariogenic streptococci using a suspended solution format. Cultures tested include Streptococcus mutans ATCC 700610, Streptococcus mutans A TCC 25175, Streptococcus sanguis and Streptococcus sobrinus. Overnight cultures were prepared using BHI medium and then inoculum was transferred to fresh BHI medium in the presence (0.5 U/ml-10 U/ml) or absence of bacitracin. The results demonstrate that 0.5-1.0 U/ml bactracin are selection doses that greatly inhibit non-cariogenic streptococci, such as S. sanguis, but allows considerable break-through of growth of cariogenic S. mutans and S. sobrinus (both members of the cariogenic mutans streptococci group). FIG. 4 illustrates the effect of sucrose as a potential accelerant or enhancer of growth of oral streptococci, which may have direct applicability in extending the effectiveness of bacitracin selection of cariogenic streptococci. These data measure the amount of bacteria suspended in the growth medium at various times following the inoculation of the culture, and demonstrates that 1) sucrose will extend the exponential phase of growth and promote increased yields at saturation for oral streptococci, including cariogenic S. mutans and non-cariogenic S. salivarius and S. sanguis, and 2) sucrose, especially at concentrations of 0.5-1%, will promote the increased adherance of S. mutans in a biofilm coating the plastic surface of the culture vessel. In the case of S. mutans, the cumulative sum of suspended bacteria and bacteria recovered from the biofilm, demonstrates a curve similar to the non-cariogenic Streptococci, that prolongs the exponential phase of growth with higher yields at saturation. This prolonged exponential phase of growth will enhance the effectiveness of bacitracin selection, which is dependent on the presence of dividing cells and hence is directly related to the duration of the exponential phase. Thus, the presence of sucrose may represent a further improvement of the current CariScreen device, by 1) extending the effectiveness of bacitracin selection and by 2) increasing the likelihood or allowing all oral microorganisms to remain in an exponential phase of growth, which based on FIG. 2, represents an accurate determinant of bacterial number as measured by ATP.

In a further embodiment,

### IV. SYSTEMS AND KITS FOR THE SELECTION AND QUANTITATION OF CARIOGENIC BACTERIA

As indicated above, provided herein are systems for the selection, detection and quantitation of cariogenic bacteria from an oral sample, such as a dental plaque and/or saliva. Within certain embodiments, systems comprise a two-stage reservoir wherein a first stage contains a selection medium such as, for example, mutacin, bactracin, and Rogosa Medium and a second stage contains a mixture comprising a lysing medium, luciferin, luciferase, and a magnesium solution.

Oral samples may be obtained, including dental plaque and/or saliva. In one embodiment a dental plaque is disrupted to facilitate accurate sampling. Because dental plaques, the bacterial film adhering to tooth surfaces, are composed of closely packed bacteria and noncellular material, such plaques can interfere with accurate quantitation of cariogenic bacteria. Roughly 20% of the dry weight of dental plaque is water-insoluble glucans, thus in one embodiment, the plaque may be disrupted, for example, by treating the plaque with an oral solution containing an amylase, mutanase and/or dextranase, for example a mutanase obtained from a microorganism belonging to the genus Bacillus having negative protease producibility, such as described in U.S. Pat. No. 5,741,487 to Asai et al. Such oral samples including one or more oral bacterial species may be sampled with a swab, which is placed into a first reservoir containing a first selection medium. Following an incubation period, typically at approximately room temperature or 37° C. although optionally at a higher temperature to accelerate the bacterial growth rate, a second stage solution is combined into the incubated dental plaque and visible light from ATP originating from selected cariogenic species is measured, for example, with a hand-held luminometer. Addition of adenosine phosphate deaminase and apyrase may, optionally, be required to eliminate extracellular ATP released from inhibited oral microorganisms.

In the case of mutacin pre-selection, an upper reservoir may also contain two independent stages. In such embodiments, a first stage may contain medium plus mutacin from Streptococcus Mutans UA159, as well as adenosine phosphate deaminase and apyrase to eliminate extracellular ATP released from mutacin-inhibited microorganisms. A second stage may contain a cell-lysing medium, luciferin, luciferase, and a magnesium solution (as described above). The addition of adensine phosphate deaminase and apyrase would not be necessary for the pre-selection using bacitracin because of the longer incubation time and resulting degradation of extracellular ATP during this treatment time.

In the case of mutacin pre-selection, the first-stage solution will, for example, contain mitis salivarius medium plus sucrose, for example from about 0.1% to about 2% and in particular about 1% sucrose (without bacitracin) and will be allowed to incubate with the oral sample. At the end of this incubation, a second-stage solution may then be drained into the first-stage solution and visible light from ATP that originates from the selected Mutans streptococci is measured, such as with a hand-held luminometer.

### V. EXAMPLES

The following examples serve to more fully describe the manner of using the above-described invention, as well as to set forth the best modes contemplated for carrying out various aspects of the invention. These examples in no way serve to limit the true scope of this invention, but rather are presented for illustrative purposes.

### Example 1

### Test Microorganisms

This Example describes additional suitable organisms for detection and quantitation using the systems, kits, and methods described herein. Selected microbiological agents are acquired from multiple sources for use in an exemplary ATP bioluminescence test. These sources include the ATCC stocks disclosed in Table 2:

TABLE 2
Exemplary Cariogenic Streptococcus and Lactobacillus Test Organisms
Available from the ATCC

| ATCC ® | |
|---|---|
| Number | Description/Designation/Select |
| 31989 | Streptococcus mutans Clarke UAB308 |
| 19641 | Streptococcus sp. HS-4 |
| 19643 | Streptococcus sp. HS-7 |
| 19644 | Streptococcus sp. HS-10 |
| 19645 | Streptococcus ratti FA-1 [CNCTC 10/89] |
| 25975 | Streptococcus salivarius |
| 27006 | Streptococcus sp. SS2 |
| 27351 | Streptococcus sobrinus deposited as |
| | Streptococcus mutans Clarke NIDR 6715-7 |
| 27607 | Streptococcus sobrinus deposited as |
| | Streptococcus mutans Clarke SL-1 |
| 31412 | Streptococcus intermedius Si-1 |
| 33478 | Streptococcus sobrinus SL1 [CCM 6070; CNCTC 9/89] |
| 35911 | Streptococcus macacae NCTC 11558 |
| 49125 | Streptococcus vestibularis PV91 [NCTC 12167] |
| 49126 | Streptococcus vestibularis HV81 |
| 49295 | Streptococcus sanguinis KTH-3 |
| 49296 | Streptococcus sanguinis KTH-2 [FERM-P 8169; MCLS-2] |
| 49297 | Streptococcus sanguinis KTH-4 |
| 49298 | Streptococcus sanguinis KTH-1 [FERM-P 7372; SSH-83] |
| 49999 | Streptococcus cristatus CC5A |
| 51100 | Streptococcus cristatus CR311 [CIP 105954; NCTC 12479] |
| 49296 | Streptococcus sanguinis KTH-2 [FERM-P 8169; MCLS-2] |
| 49297 | Streptococcus sanguinis KTH-4 |
| 49298 | Streptococcus sanguinis KTH-1 [FERM-P 7372; SSH-83] |
| 49299 | Streptococcus cristatus CC5A |
| 51100 | Streptococcus cristatus CR311 [CIP 105954; NCTC 12479] |
| 51656 | Streptococcus gordonii VPI E1A-1A [PK488] |
| 55229 | Streptococcus oralis NIH strain H1 |
| 55676 | Streptococcus mutans JH 1140 |
| 55677 | Streptococcus mutans JH 1000 |
| 700233 | Streptococcus oralis VPI D208B-16 |
| 700234 | Streptococcus oralis VPI D284B-05 |
| 700611 | Streptococcus mutans UA130 [US130S; UAB576] |
| 700640 | Streptococcus orisratti A63 |
| 700641 | Streptococcus australis AI-1 |
| 51655 | Actinomyces naeslundii PK606 [RC29] |
| 11577 | Lactobacillus buchneri deposited as Lactobacillus brevis |
| | (Orla-Jensen) Bergey et al. |
| 11578 | Lactobacillus casei deposited as Lactobacillus casei subsp. |
| | casei (Orla-Jensen) Hansen and Lessel |
| 11579 | Lactobacillus buchneri |
| 11582 | Lactobacillus paracasei subsp. paracasei deposited as |
| | Lactobacillus casei (Orla-Jensen) Hansen and Lessel [NCDO |
| | 680] |
| 11739 | Lactobacillus fermentum deposited as Lactobacillus |
| | cellobiosus Rogosa et al. 19LC3 [P. A. Hansen L 872] |
| 11740 | Lactobacillus fermentum deposited as Lactobacillus |
| | cellobiosus Rogosa et al. 19LC3 [P. A. Hansen L 872] |
| 11741 | Lactobacillus salivarius subsp. salivarius HO66 |
| 11742 | Lactobacillus salivarius subsp. salicinius HO268 |
| 12935 | Lactobacillus buchneri L869 [474] |
| 12936 | Lactobacillus buchneri L870 [708B] |
| 14932 | Lactobacillus fermentum L888 |
| 49062 | Lactobacillus oris NCDO 2160 [1978; 5A1; NCIB 8831] |
| 49627 | Olsenella uli deposited as Lactobacillus uli Olsen et al. PI |
| | D76D-27C [NCFB 2895] |
| BAA-793 | Lactobacillus plantarum NCIMB 8826 [Hayward 3A] |
| 27872 | Capnocytophaga ochracea deposited as Bacteroides |
| | ochraceus (Prevot) Holdeman and Moore VPI 2845 [SS31] |
| 7469 | Lactobacillus rhamnosus deposited as Lactobacillus casei |
| | (Ora-Jensen) Holland [UCSAV 227; M. Rogosa v300; |
| | M. E. Sharpe H2; NCDO 243; NCIB 6375; |
| | NCIB 8010; NCTC 6375; NRC 488; |
| | P. A. Hansen 300; R. P. Tittsler 300] |

### Example 2

This example describes the determination of doubling rate, lag time, initiation of stationary phase, and determination of growth levels at saturation for several oral streptococci and lactobacilli. In all cases, the source of inoculum was from an overnight culture dispensed into 75 ml of BHI medium and grown at 37° C. in a shaker incubator in the presence of supplemental 5% CO₂. The results are recorded in Table 3.

TABLE 3
Doubling rate, lag time, initiation of stationary phase, and determination of
growth levels at saturation for several oral streptococci and lactobacilli

| | | | Stationary | |
|---|---|---|---|---|
| | | | Phase | |
| | Doubling | Lag Period | (initiation | OD Saturation |
| | Rate | (duration in | point in | (Absorbance |
| Species | (minutes) | minutes) | minutes) | at 600 nm) |
| S. mutans | 96 | 120 | 390 | 0.675 |
| 700610 | | | | |
| S. mutans | 89 | 120 | 390 | 0.685 |
| 700610 | | | | |
| S. mutans | 71.8 | 120 | 360 | 0.715 |
| 25175 | | | | |
| S. mutans | 77.2 | 120 | 360 | 0.702 |
| 25175 | | | | |
| S. sanguis | 99.3 | 120 | 390 | 0.425 |
| S. sanguis | 95.7 | 120 | 390 | 0.43 |
| S. gordonii | 67 | 240 | 390 | 0.182 |
| S. gordonii | 57 | 270 | 390 | 0.197 |
| S. salivarius | 30 | 60 | 180 | 0.74 |
| S. salivarius | 30 | 60 | 180 | 0.729 |
| S. sobrinus | 57.3 | 120 | 360 | 0.64 |
| S. sobrinus | 60.8 | 120 | 360 | 0.67 |

### Example 3

### A System for Assaying Microorganisms Associated with Dental Caries Formation

This example describes a system and method Microorganisms are collected from an oral sample using a sterile swab which is subsequently inserted into the bottom of a plastic tube. The tube is molded with an upper reservoir holding a cell-lysis solution and containing luciferin, luciferase and Mg⁺². A plastic seal is broken allowing the solution in the upper reservoir to drain into the bottom of the plastic tube and to contact the swab in the bottom of the plastic tube. Microorganisms from the swab and the luciferin-containing solution are gently mixed for 20 seconds. The quantity of visible light released from the luciferin reaction, driven by the ATP originating from the collected microorganisms, is then measured using a hand-held luminometer. Reagents for measuring ATP are via the luciferin luciferase assay are available, for example, from BioThema AB, Stationsvägen, Sweden.

### Example 4

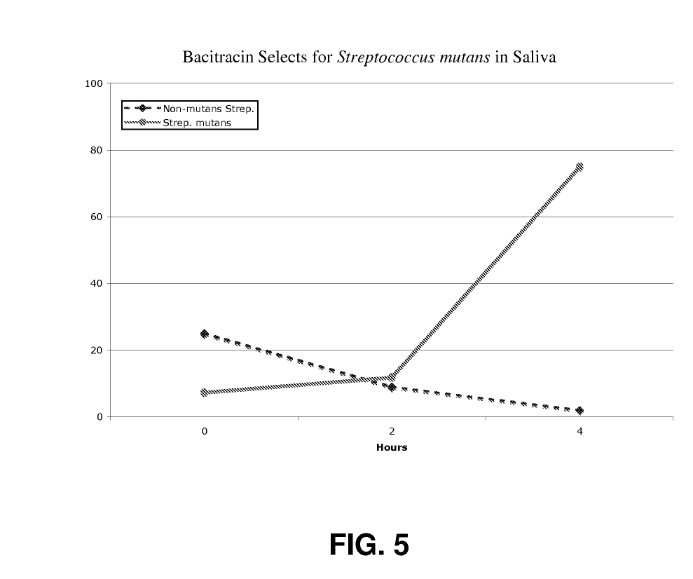

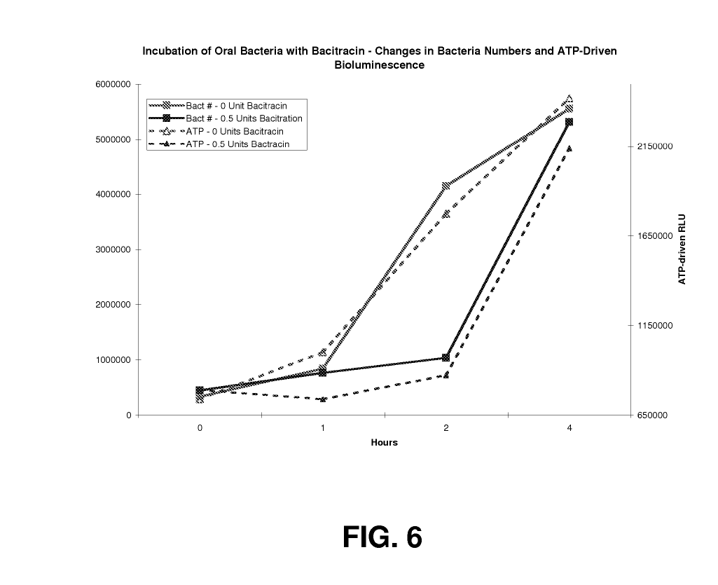

This example describes a clinical study, examining the application of bacitracin selection (and sucrose growth accelerant) on oral micro-organisms found in dental plaque and saliva. This study examines up to 50 patients, with 3-4 plaque specimens and one saliva specimen per patient, and assesses the correlation between ATP content (measured by CariScreen meter and Veritas luminometer), wet weight of plaque specimens, total protein measurement, and viable bacterial numbers (total bacteria, total oral streptococci, Streptococcus mutans, and total lactobacilli as assessed with the use of plating on blood agar, mitis-salivarius agar, mitis-salivarius agar supplemented with bacitracin (0.2% w/v), and Rogasa agar, respectively). FIG. 5, which includes initial data from this study, demonstrates that incubating pediatric saliva samples in media (BHI) plus bacitracin selects for S. mutans species. With continued reference to FIG. 5, with four hours of bacitracin treatment the percentage of S. mutans in saliva went from 7% at time 0 to 75%. With reference to FIG. 6, a subject's saliva was added to BHI media±bacitracin (0.5 units/ml), and then incubated at 37° C. in a 5% carbon dioxide atmosphere. At times 0, 1, 2, and 4 hours samples were measured for ATP-driven bioluminescence and plated onto blood agar plates to measure total bacteria numbers. The upper solid and hashed lines in FIG. 6 chart population growth and ATP signal, respectively, in the absence of bacitracin, and the lower solid and hashed lines chart the same in the presence of a selection agent comprising bacitracin. FIG. 6 demonstrates that ATP-driven bioluminescence can be used to measure these S. mutans selected from a diverse bacteria population, the selection being detectable within four hours.

### Example 5

This example describes one embodiment of preparing a calibration curve of ATP used in examples was prepared by the following procedure. The measurement of ATP is carried out by adding a luciferin-luciferase containing luminescent reagent to an ATP-containing sample and quantitatively determining the amount of bioluminescence released. The reagent kit for measuring ATP as a luminescence amount with the luciferin-luciferase containing luminescent reagent and the apparatus for measuring the luminescence amount are commercially available, for example, from BioThema AB, Stationsvägen, Sweden. Such reagents and apparatus also are available from PerkinElmer, Boston, Mass.

An example of the luciferin-luciferase containing luminescent reagent (reagent for measuring ATP) (see Bunseki Kagaku, 1995, 44, 845-851), contains the following ingredients: 10 mM magnesium sulfate (Mg²⁺ ion),
0.30 mM D-luciferin (luminescent material),
1.0 mM EDTA (stabilizer),
1.0 mM dithiothreitol (stabilizer),
0.51 mg/mL luciferase (from Genji firefly) (luminescent enzyme),
0.2% bovine serum albumin (BSA) (stabilizer),
in 50 mM HEPES buffer (pH 7.8).

An exemplary preparation of the calibration curve of ATP with a luminometer “Lumat LB9501” manufactured by Berthold is described below. Water (100 μl) is added to 100 μl of an ATP standard solution having a known concentration, followed by 100 μl of a luciferin-luciferase containing luminescent reagent (luminescent reagent) to estimate the relative light Unit S by the measurement of luminescence after a one second lag time before integration for three seconds with the luminometer LB9501. At the same time, 100 μl of a luciferin-luciferase containing luminescent reagent is added to 200 μl of water to carry out the measurement of luminescence for estimate the luminescence amount R in the same manner as above. The measurement R is used as the blank. The difference S-R gives the net amount of luminescence of ATP (Z). An calibration curve of ATP can prepared by setting the Y axis of the coordinates as the net amount of luminescence Z and the X axis as the ATP concentration (M=mole/L) as shown in FIG. 6.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A method for assessing risk of dental caries by quantitating cariogenic bacteria, comprising obtaining an oral sample that contains oral bacteria from a subject;
selecting for cariogenic bacteria in the oral sample; and
quantifying cariogenic bacteria in the sample, wherein quantifying comprises measuring ATP in the oral sample, and an amount of ATP in the sample predicts a quantity of the selected cariogenic bacteria.

2. The method of claim 1, wherein the oral sample comprises dental plaque, saliva or both.

3. The method of claim 1, wherein obtaining the oral sample comprises contacting dental plaque with an amylase, dextranase, mutanase or a combination thereof.

4. The method of claim 1, wherein selecting comprises contacting the oral sample with a growth medium selective for cariogenic bacteria.

5. The method of claim 4, wherein selecting comprises contacting the oral sample with a medium comprising an antibiotic that selectively inhibits the growth of non-cariogenic bacteria.

6. The method of claim 5, wherein selecting comprises contacting the oral sample with the medium comprising an antibiotic for less than about four hours.

7. The method of claim 5, wherein selecting comprises contacting the oral sample with the medium comprising an antibiotic for from about 20 minutes to about two hours.

8. The method of claim 5, wherein selecting comprises contacting the oral sample with the medium comprising an antibiotic for from about 12 hours to about 24 hours.

9. The method of claim 5 wherein the antibiotic is selected from the pore forming antibiotics.

10. The method of claim 5, wherein the antibiotic is a mutacin or bacitracin.

11. The method of claim 4, wherein selecting comprises contacting said oral sample with Rogosa Medium.

12. The method of claim 4, further comprising contacting the oral sample with a bacterial growth accelerant.

13. The method of claim 12, wherein the growth accelerant comprises sucrose.

14. The method of claim 12, wherein the growth accelerant comprises a biofilm.

15. The method of claim 1 wherein the cariogenic bacteria includes a Streptococcus or Lactobacillus bacterium.

16. The method of claim 15 wherein Streptococcus is Streptococcus mutans or Streptococcus sobrinus.

17. The method of claim 15, wherein the Lactobacillus is Lactobacillus casei.

18. The method of claim 1 wherein quantifying comprises detecting and quantifying ATP using bioluminescence, wherein a detected intensity of bioluminescence is correlated with a quantity of cariogenic bacteria.

19. The method of claim 1, further comprising selecting a subject for anti-caries treatment if a quantity of selected cariogenic bacteria is above a pre-selected threshold.

20. A kit for the detection of cariogenic bacteria, comprising:
a first solution comprising a selection agent for selecting cariogenic bacteria in an oral sample;
a second solution comprising a cell-lysis solution, luciferin, luciferase and Mg⁺²;
a first chamber containing the first solution; and a second chamber containing the second solution.

21. The kit of claim 20, further comprising a reservoir for mixing the first and second solutions.

22. The kit of claim 20, wherein the first solution further comprises a bacterial growth accelerant.

23. A system for the quantitation of cariogenic bacteria, comprising:
a first solution comprising an agent for selecting for cariogenic bacteria in an oral sample;
a second solution comprising a cell-lysis solution, luciferin, luciferase and Mg⁺²,
a first chamber and a second chamber wherein said first solution is in said first chamber and said second solution is in said second chamber; and an instrument for detecting light emitted when said first solution is mixed with said second solution in the presence of said cariogenic bacterium.

24. The system of claim 19, wherein the instrument for detecting light is a luminescence biometer.

25. A method for evaluating the efficacy of an oral care product in the reduction of cariogenic bacteria, comprising:
obtaining a first oral sample from a subject;
quantifying cariogenic bacteria in the first oral sample, wherein quantifying comprises measuring ATP in the first oral sample;
providing the subject with the oral care product;
obtaining a second oral sample from the subject;
quantifying cariogenic bacteria in the second oral sample, wherein quantifying comprises measuring ATP in the second oral sample; and
comparing the quantity of cariogenic bacteria in the first and second oral samples.

26. The method of claim 25, wherein the oral care product comprises a fluoride rinse.
